(19) United States
(12) Patent Application Publication
(10) Pub. No.: US 2026/0253693 A1
(43) Pub. Date: Aug. 27, 2026

(54) SYSTEM FOR CONTEXT-AWARE INFORMATION DELIVERY WITHIN ELECTRONIC HEALTH RECORD WORKFLOWS

(71) Applicant: HARSHIT JAIN, SHORT HILLS, NJ (US)

(72) Inventor: HARSHIT JAIN, SHORT HILLS, NJ (US)

(21) Appl. No.: 19/650,088

(22) Filed: Apr. 16, 2026

Related U.S. Application Data
(63) Continuation-in-part of application No. 18/395,123, filed on Dec. 22, 2023, now Pat. No. 12,254,489.

(51) Int. Cl. G16H 10/60; G06Q 10/10

(52) U.S. Cl. CPC G16H 10/60; G06Q 10/103

(57) ABSTRACT

The embodiments disclose a system is disclosed for integrating regulated life sciences information into an electronic health record (EHR) workflow including one or more processors, non-transitory memory storing executable instructions, and an interface configured to communicate with at least one EHR system, during an active clinical workflow, the system authenticates a healthcare provider accessing a patient record and extracts one or more clinical triggers from the record, based on the extracted clinical triggers, the system selects at least one information object from a plurality of regulated life sciences information objects stored in a database, the selected information object is presented within a predefined region of an EHR workflow interface in a manner that does not interrupt the clinical workflow.

### CROSS-REFERENCE TO RELATED APPLICATIONS

This Patent Application is a continuation-in-part of and claims priority to United States Patent Application entitled: “HCP CONSENT MANAGEMENT FRAMEWORK SYSTEM”, U.S. Ser. No. 18/395,123 filed on Dec. 22, 2023, by Harshit Jain, which is a Continuation in-part of United States Patent Application entitled: “UNIFIED DYNAMIC OBJECTS GENERATED FOR WEBSITE INTEGRATION”, U.S. Ser. No. 18/379,056 filed on Dec. 22, 2023, by Harshit Jain, which is a Continuation in-part of United States Patent Application entitled: “ELECTRONIC HEALTH RECORD PLATFORM”, U.S. Ser. No. 18/116,290 filed on Mar. 1, 2023, by Harshit Jain, which is a Continuation in-part of United States Patent Application entitled: “ELECTRONIC MEDICAL RECORD ADVERTISING PLATFORM METHOD AND DEVICES”, U.S. Ser. No. 17/857,013 filed on Jul. 3, 2022, by Harshit Jain, all of which applications are incorporated herein by reference.

### BACKGROUND

Healthcare information systems increasingly rely on electronic health record (EHR) platforms to support clinical workflows, documentation, prescribing, and decision-making by healthcare professionals. Modern EHR environments present complex technical challenges related to the integration of supplemental clinical and life sciences information within active patient-care workflows, while maintaining strict compliance with patient privacy regulations and healthcare data governance requirements.

Healthcare professionals are required to access a broad range of clinical, therapeutic, and life sciences information during patient encounters, including treatment guidelines, disease awareness materials, affordability programs, and medication-related data. However, existing systems often require clinicians to leave the EHR workflow to search for or retrieve such information from external systems, resulting in workflow disruption, increased cognitive load, and reduced efficiency in patient care delivery.

Conventional approaches for presenting external information within EHR systems suffer from significant technical limitations. These approaches frequently lack real-time integration with clinical context, fail to account for patient-specific attributes in a compliant manner, and do not provide a scalable mechanism for dynamically selecting and presenting relevant information within predefined EHR workflow interfaces. Additionally, many existing solutions are not designed to operate within the regulatory constraints imposed by healthcare privacy frameworks such as HIPAA, GDPR, and similar regional regulations.

Another challenge arises from the need to coordinate information delivery across heterogeneous EHR platforms, devices, and healthcare environments. Healthcare professionals may interact with EHR systems through multiple interfaces and devices, requiring consistent enforcement of access controls, content eligibility rules, and compliance policies. Existing systems often lack the architectural capability to manage such coordination in a unified and compliant manner.

Furthermore, healthcare information systems must support auditability, transparency, and traceability of information presentation within clinical workflows. Regulatory bodies, healthcare institutions, and system operators require mechanisms to verify that information delivery complies with applicable policies, consent requirements, and usage constraints. Many prior systems do not provide sufficient logging, audit trails, or reporting mechanisms to satisfy these requirements.

Accordingly, there exists a need for an improved system and platform that enables compliant, context-aware integration of supplemental information within electronic health record workflows. Such a system should support real-time data processing, rule-based content selection, secure platform architecture, and regulatory compliance, while minimizing disruption to clinical workflows and preserving the integrity of patient-care environments.

### SUMMARY OF THE INVENTION

The present invention relates to systems and platforms for integrating consent-compliant information delivery within electronic health record (EHR) workflows. In one embodiment, the invention provides a distributed system architecture configured to deliver supplemental clinical and life sciences information to healthcare professionals directly within active EHR interfaces, while maintaining compliance with applicable healthcare privacy and data protection regulations.

In one embodiment, the system includes a network platform comprising one or more processors, memory devices, and databases configured to store clinical trigger data, content rules, and regulated information objects. The system is further configured to interface with one or more EHR systems to extract patient privacy-compliant clinical signals during an active clinical workflow. Based on the extracted signals, the system dynamically selects and presents relevant information within predefined EHR workflow pages without requiring a healthcare professional to exit the clinical environment.

In another embodiment, the system supports real-time content selection and integration using rule-based processing, bid-based selection logic, or prioritization mechanisms executed by the network platform. Selected content is embedded into EHR workflow interfaces using graphical rendering components, ensuring continuity of clinical workflows and minimizing disruption during patient care activities.

In a further embodiment, the system supports distributed operation across multiple EHR platforms and healthcare environments. The system enforces access controls, consent policies, and regulatory constraints consistently across devices, sessions, and institutional boundaries. Audit logging and compliance verification mechanisms are provided to support transparency, traceability, and regulatory oversight.

In yet another embodiment, the system includes tracking and analytics components configured to associate information presentation events with healthcare professional interactions within EHR workflows. Aggregated interaction data may be processed to generate system-level metrics and reports for platform operators and authorized entities, while preserving patient privacy and adhering to applicable legal and regulatory requirements.

Advantageously, the disclosed system enables compliant, context-aware delivery of supplemental information within EHR workflows, improves workflow efficiency for healthcare professionals, and provides a scalable technical infrastructure for regulated information integration within healthcare information systems.

In certain embodiments, the present disclosure relates to systems and methods implemented using one or more computing systems comprising tangible, physical hardware components including one or more processors, processing circuits, memory devices, communication interfaces, and one or more input and output devices configured to acquire, sense, measure, detect, transform, process, analyze, and generate data representative of one or more physical, environmental, biological, mechanical, or user-associated states. The computing systems may include, without limitation, wearable devices, mobile devices, smartphones, tablet computers, desktop computers, laptop computers, embedded systems, edge computing devices, Internet-of-Things (IoT) devices, smart home devices, automotive systems, industrial control systems, servers, cloud computing platforms, and combinations or distributed arrangements thereof. In certain embodiments, the processors may include one or more central processing units (CPUs), graphics processing units (GPUs), tensor processing units (TPUs), digital signal processors (DSPs), field-programmable gate arrays (FPGAs), application-specific integrated circuits (ASICs), or system-on-chip (SoC) architectures configured to execute machine-readable instructions and perform signal processing and data transformation operations.

The input devices may include one or more sensors and data acquisition components configured to generate electrical, optical, acoustic, electromagnetic, or digital signals corresponding to measurable phenomena. Such sensors may include, without limitation, physiological sensors, biometric sensors, environmental sensors, optical sensors, imaging devices such as cameras or scanners, depth sensors, infrared sensors, audio sensors such as microphones, motion sensors such as accelerometers, gyroscopes, or inertial measurement units (IMUs), proximity sensors, pressure sensors, location sensors such as global positioning system (GPS) modules, radio-frequency identification (RFID) readers, or other sensing devices configured to capture data associated with a user, an object, or an environment. The output devices may include displays, graphical user interfaces (GUIs), speakers, haptic feedback devices, actuators, control systems, or other devices configured to present information or initiate physical actions in response to generated outputs.

The communication interfaces may include wired or wireless transceivers configured to communicate data between devices over one or more communication networks, including local area networks (LANs), wide area networks (WANs), cellular networks, satellite networks, Internet-based networks, or short-range communication protocols including WiFi, Bluetooth, Bluetooth Low Energy (BLE), near-field communication (NFC), ultra-wideband (UWB), Zigbee, or other radio-frequency communication systems. In certain embodiments, communication may be facilitated using network identifiers including Internet Protocol (IP) addresses, media access control (MAC) addresses, device identifiers, session identifiers, or other addressing schemes for routing, synchronization, and coordination of data transmissions.

The computing systems may be configured to receive raw input signals from the input devices and to perform one or more physical and computational transformations on such signals. Such transformations may include analog-to-digital conversion, signal conditioning, filtering, denoising, baseline correction, normalization, scaling, time alignment, synchronization across multiple data streams, segmentation into discrete time intervals or data structures, feature extraction, encoding into vector representations, dimensionality reduction, compression, or other data transformation operations that convert raw sensor or input data into structured, machine-interpretable data representations. These structured representations may correspond to time-series data, spatial data, image data, audio data, or multi-modal data.

The structured data may then be processed using one or more computational models, including deterministic algorithms, statistical models, rule-based systems, or artificial intelligence and machine learning models, to generate outputs that correspond to a practical application. Such outputs may include classifications, detections, identifications, predictions, correlations, anomaly detections, comparisons with stored data, control signals for external systems, alerts, recommendations, or other actionable outputs that are used to control, modify, or influence a physical system, user interface, or decision-making process.

The present invention utilizes multiple mathematical models configured to convert continuous egocentric sensor streams into structured spatiotemporal world models and to optimize data retention under resource constraints. In one embodiment, the system represents an environment using a spatiotemporal world graph model defined as G=(N, E, T, M), wherein N represents nodes corresponding to objects, agents, and interactions, E represents edges corresponding to causal, temporal, spatial, and containment relationships, T represents temporal state trajectories, and M represents metadata including confidence, informational yield, and data provenance. The system further includes an object identity association model configured for multi-factor identity association, wherein identity association is computed using appearance similarity, spatial relationship functions, motion functions, and class consistency indicators to maintain persistent identity tracking across occlusion events and across multiple sessions.

In one embodiment, the system is further configured to optimize informational yield per unit energy using an informational yield per joule model defined as the ratio between uncertainty reduction and energy consumed in acquiring data. The system further includes an entropy-driven retention model in which data retention decisions are determined as a function of system entropy, battery state, thermal state, and compute load. The system additionally computes a composite learning yield score as a weighted function of uncertainty reduction, novelty, transition density, and coverage gain, wherein said composite learning yield score is used to prioritize data retention, processing, and learning operations.

The system utilizes multiple algorithm classes to implement perception, tracking, interaction detection, segmentation, yield estimation, retention optimization, scheduling, graph construction, distributed learning, and data compression. Visual perception is performed using Convolutional Neural Networks (CNN) and Vision Transformers. Object identity persistence is maintained using multi-factor data association and Bayesian filtering. Motion tracking is performed using Visual-Inertial Odometry (VIO) and Kalman filtering. Interaction detection is performed using state transition detection and velocity coupling detection algorithms. Segmentation is performed using event boundary detection algorithms. Yield scoring is performed using active learning and uncertainty estimation algorithms. Retention policy optimization is performed using reinforcement learning and optimization algorithms. Scheduling is performed using resource-aware adaptive scheduling algorithms. Graph construction is performed using incremental graph building algorithms. Federated learning is performed using distributed gradient aggregation. Mission detection is performed using semantic embedding similarity matching. Retroactive reprocessing is performed using a versioned data reprocessing pipeline. Compression is performed using delta encoding and embedding compression.

In one embodiment, the invention operates on a hardware system comprising one or more sensors, compute units, memory units, and power monitoring components. Sensors may include RGB cameras, stereo cameras, depth sensors, inertial measurement units (IMU), microphone arrays, eye tracking sensors, and wrist-mounted IMU or haptic sensors. Compute hardware may include one or more CPUs for general processing, GPUs for neural network inference, TPUs or NPUs for AI acceleration, DSPs for signal processing, and FPGA or ASIC hardware for low-power inference. The system further includes on-device memory configured to store graph structures and associated data and a battery and power monitoring system configured to measure energy consumption for informational yield per joule calculations.

The artificial intelligence system performs multiple functional roles within the invention, including detecting and tracking objects and generating egocentric video, maintaining persistent identity of objects across occlusion and across sessions, detecting interaction boundaries based on physical state transitions, constructing and updating a spatiotemporal world graph, estimating uncertainty and learning value of observed interactions, computing informational yield per joule for interaction segments, selecting data for retention or deletion based on yield optimization, adjusting sensing and compute fidelity based on thermal state, battery state, and compute load, learning improved retention policies using reinforcement learning and federated learning, detecting task or mission completion using semantic pattern matching, retroactively reprocessing stored data using improved AI models, and producing structured datasets for training robotic and physical AI systems.

The artificial intelligence system and hardware components operate together as a closed-loop control system in which sensor data is processed by AI perception modules, which update the spatiotemporal graph model, which feeds yield scoring and retention decision modules, which feed a scheduler that adjusts sensor and compute settings, which in turn modifies subsequent sensor data acquisition. This closed-loop learning optimization system maximizes learning value per unit of energy on a wearable device.

### BRIEF DESCRIPTION OF THE DRAWINGS

FIG. 1 shows, for illustrative purposes only, an example of an overview of an electronic medical record advertising platform of one embodiment.

FIG. 2 shows a block diagram of an overview flow chart of targeting life science advertiser/supplier products of one embodiment.

FIG. 3 shows a block diagram of an overview of a winning bid selection processor of one embodiment.

FIG. 4 shows a block diagram of an overview of an electronic medical record advertising platform of one embodiment.

FIG. 5 shows, for illustrative purposes only, an example of a patient calendar of one embodiment.

FIG. 6 shows, for illustrative purposes only, an example of a patient chart of one embodiment.

FIG. 7 shows a block diagram of an overview of providing a physician with information of one embodiment.

FIG. 8 shows a block diagram of an overview of the creation of ad copy in various formats of one embodiment.

FIG. 9 shows a block diagram of an overview of providing the latest medical information to health care providers of one embodiment.

FIG. 10 shows a block diagram of an overview of patient affordability messaging of one embodiment.

FIG. 11 shows a block diagram of an overview of clinical alerts of one embodiment.

FIG. 12 shows a block diagram of an overview of disease awareness messaging of one embodiment.

FIG. 13 shows a block diagram of an overview of ads and information integrated within the practice workflow of one embodiment.

FIG. 14 shows a block diagram of an overview of selecting a precise message based on the workflow of one embodiment.

FIG. 15 shows a block diagram of an overview of outcomes of one embodiment.

FIG. 16 shows a block diagram of an overview of HIPAA compliance of one embodiment.

FIG. 17 shows a block diagram of an overview of engaging with health care providers of one embodiment.

FIG. 18 shows a block diagram of an overview of an onboarding process of one embodiment.

FIG. 19 shows a block diagram of an overview of partnering of one embodiment.

FIG. 20 shows a block diagram of an overview of targeted EHR webpages to bid on of one embodiment.

FIG. 21 shows a block diagram of an overview of establishing health care provider workflow web pages of one embodiment.

FIG. 22 shows a block diagram of an overview of physician e-prescribing medication from a product advertiser of one embodiment.

### DETAILED DESCRIPTION OF THE INVENTION

In the following description, reference is made to the accompanying drawings, which form a part hereof, and in which is shown by way of illustration a specific example in which the invention may be practiced. It is to be understood that other embodiments may be utilized, and structural changes may be made without departing from the scope of the present invention.

It should be noted that the descriptions that follow, for example, in terms of an electronic medical record (EMR) integrated information delivery system and associated devices, are described for illustrative purposes. The underlying system may be applied to multiple types of clinical triggers and healthcare information workflows. In one embodiment of the present invention, the system is configured for integration with electronic health record (EHR) systems and operates using clinical triggers derived from patient records. The system may be configured to present clinical alerts, disease awareness information, and other regulated healthcare information directly within EHR workflows. As used herein, a clinical trigger refers to a specific event, condition, or data element within an EHR that initiates a system response.

The terms “health care provider,” “provider,” “physician,” “doctor,” “Dr. ,” “nurses,” other healthcare worker titles, and “user” or “users” are used interchangeably herein without any change in meaning.

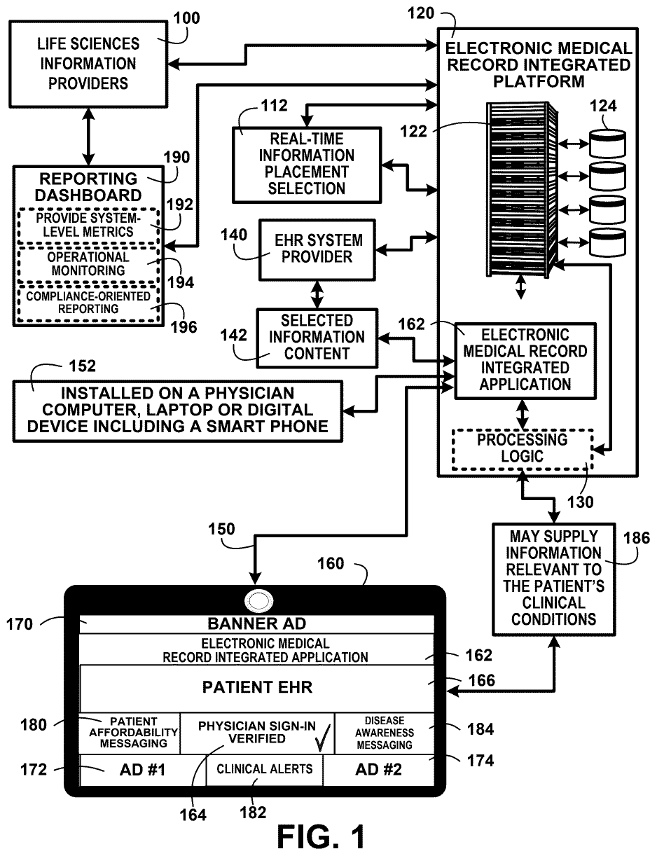

FIG. 1 shows, for illustrative purposes only, an example of an overview of an electronic medical record integrated information platform of one embodiment. FIG. 1 shows, in one embodiment, life sciences information providers 100 participating in real-time information placement selection 112 through an electronic medical record integrated platform 120. An electronic medical record integrated platform 120 comprises a plurality of servers 122, a plurality of databases 124, a computing system executing an electronic medical record integrated application 162, and processing logic 130 configured to analyze clinical data. In one embodiment, selection of information content 142 for presentation within an EHR system is performed by cooperation between an electronic health record (EHR) system provider 140 and an electronic medical record integrated platform 120.

In one embodiment, the selected information content 142 is transmitted to a physician 150 through the electronic medical record integrated platform 120. In one embodiment, the selected information content 142 is transmitted through the electronic medical record integrated application 162 to a physician's digital tablet 160 during an active clinical workflow. In one embodiment, a verified physician login to the electronic medical record integrated platform 120 using the electronic medical record integrated application 162 on the physician tablet 160, displaying a patient EHR 166. In other embodiments, the electronic medical record integrated application 162 may be installed on a physician's computer, laptop, or mobile digital device, including a smartphone 152, enabling review of patient EHR data, clinical triggers, laboratory results, disease awareness information, patient affordability information 180, clinical alert 182, prescription-related data, and electronic prescribing functions.

The EHR system webpages may display informational content areas, including a banner ad 170 and additional content regions 172, 174, integrated within the workflow interface. The physician may review patient affordability information 180 presented on the physician tablet 160 when selected by the physician. The selection of a clinical alert 182 interface provides the physician with updated clinical considerations relevant to diagnosis or treatment.

Both the physician and the patient may be provided with disease awareness information 184 to support understanding of available treatment options, emerging therapies, and potential side effects associated with medications. The life sciences information providers 100 may supply information relevant to the patient's clinical conditions 186 so that the presented information corresponds to the current clinical context.

The electronic medical record integrated platform 120 may track and record presentation events associated with EHR system webpages, informational content interactions, and prescription-related actions. Aggregated tracking data may be made available through the electronic medical record integrated application 162 via a reporting dashboard 190 to provide system-level metrics 192, operational monitoring 194, and compliance-oriented reporting 196 associated with information presentation within EHR workflows.

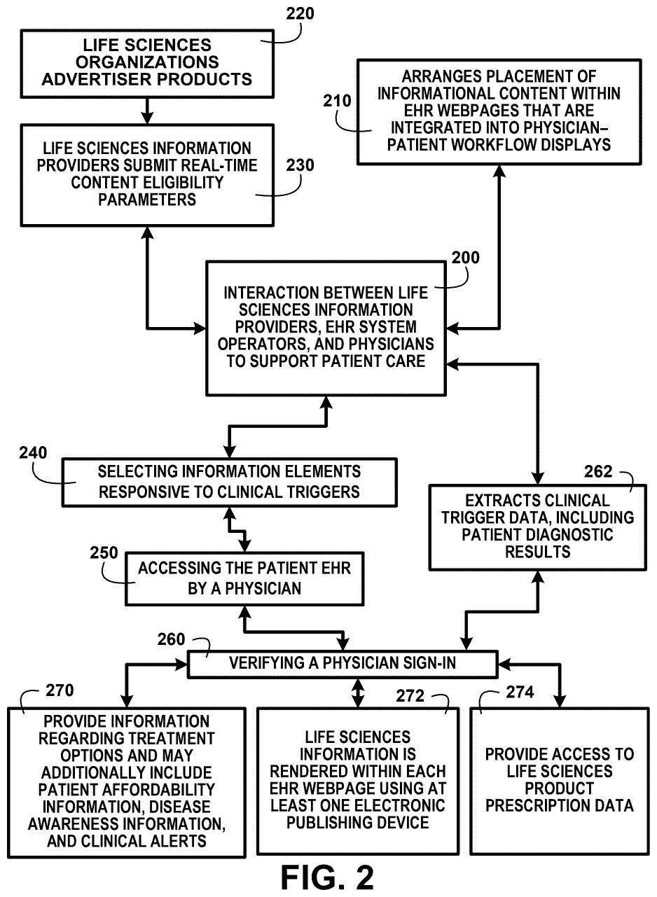

FIG. 2 shows a block diagram of an overview flow chart of targeting life science advertiser/supplier products of one embodiment. FIG. 2 illustrates a coordinated interaction between life sciences information providers, EHR system operators, and physicians to support patient care 200 through an electronic medical record integrated platform 120 of FIG. 1. The electronic medical record integrated platform 120 of FIG. 1 arranges placement of informational content within EHR webpages that are integrated into physician-patient workflow displays 210. Life sciences information associated with patient conditions is selectively presented to healthcare providers in response to detected clinical triggers.

Life sciences organization advertiser products 220 presented by life sciences information providers submit real-time content eligibility parameters 230 for inclusion within EHR workflows. An EHR system provider and the electronic medical record integrated platform 120 of FIG. 1 cooperate to select information elements responsive to clinical triggers 240. The selected information is displayed when a physician accesses a patient EHR 250. The EHR system verifies a physician sign-in 260 and, through the electronic medical record integrated platform 120 of FIG. 1, extracts clinical trigger data, including patient diagnostic results 262, while safeguarding patient privacy in compliance with the Health Insurance Portability and Accountability Act of 1996 (HIPAA).

Displayed information includes integrated content elements that provide information regarding treatment options and may additionally include patient affordability information, disease awareness information, and clinical alerts 270. Life sciences information is rendered within each EHR webpage 272 using at least one electronic publishing device. The system may also provide access to life sciences product prescription data 274 that a physician can utilize when prescribing medications for a patient in one embodiment.

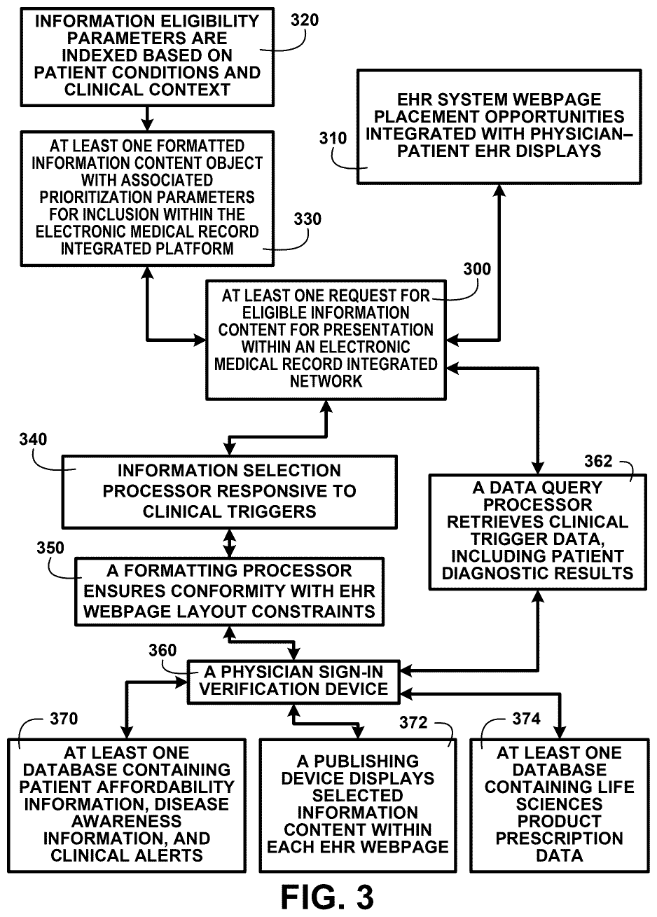

FIG. 3 shows a block diagram of an overview of a winning bid selection processor of one embodiment. FIG. 3 illustrates at least one request for eligible information content for presentation within an electronic medical record integrated network 300. The requests correspond to EHR system webpage placement opportunities integrated with physician-patient EHR displays 310. Information eligibility parameters are indexed 320 based on patient conditions and clinical context.

Life sciences information providers submit at least one formatted information content object with associated prioritization parameters for inclusion within the electronic medical record integrated platform 330. An information selection processor responsive to clinical triggers 340 analyzes the submitted content. A formatting processor 350 ensures conformity with EHR webpage layout constraints. A physician sign-in verification device 360 confirms that a physician is licensed and authorized to access the EHR system.

A data query processor retrieves clinical trigger data 362, including patient diagnostic results, from EHR records and queries at least one database containing patient affordability information, disease awareness information, and clinical alerts 370. A publishing device displays selected information content within each EHR webpage 372, including patient-condition-specific information retrieved from at least one database containing life sciences product prescription data 374, in one embodiment.

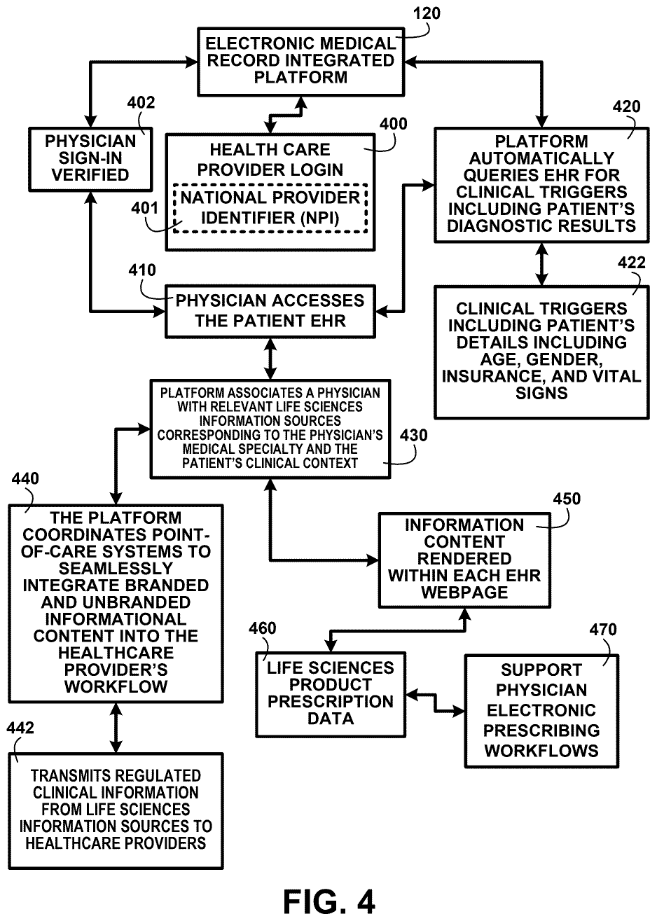

FIG. 4 shows a block diagram of an overview of an electronic medical record advertising platform of one embodiment. FIG. 4 shows an electronic medical record integrated platform 120 that interfaces with an EHR point-of-care platform to deliver regulated life sciences information within clinical workflows. A healthcare provider login 400 includes verification data such as a physician national provider identifier (NPI) 401 to authenticate access by a physician 402 to the EHR point-of-care platform, which automatically queries the EHR for clinical triggers, including clinical triggers 420, including patient diagnostic results as the physician accesses the patient EHR 410.

Clinical triggers include patient attributes such as age, gender, insurance information, and vital signs 422. The platform associates a physician with relevant life sciences information sources corresponding to the physician's medical specialty and the patient's clinical context 430. The platform coordinates point-of-care systems to seamlessly integrate branded and unbranded informational content into the healthcare provider's workflow 440. The platform transmits regulated clinical information from life sciences information sources to healthcare providers 442. Information content rendered within each EHR webpage 450 may include life sciences product prescription data 460 to support physician electronic prescribing workflows 470, of one embodiment.

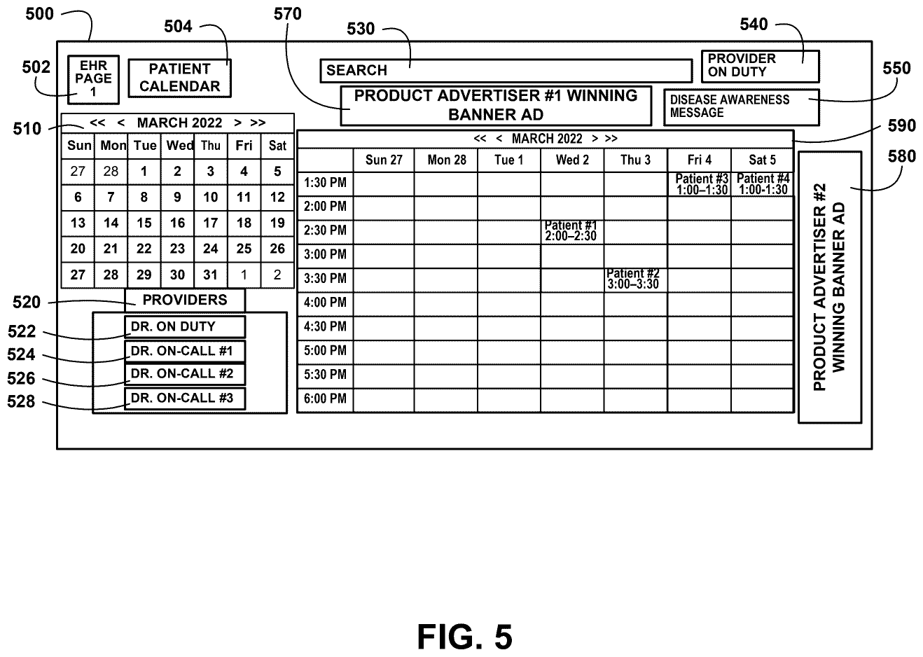

FIG. 5 shows, for illustrative purposes only, an example of a patient calendar of one embodiment. FIG. 5 shows, for example, a physician workflow webpage 500 corresponding to an EHR page 1 502 displaying a patient calendar 504. The patient calendar 504 shows a current month view 510 and a list of point-of-care providers 520, including a physician on duty 522, a physician on call #1 524, a physician on call #2 526, and a physician on call #3 528. A search interface 530 allows a physician on duty 540 to search patient EHR records.

In one embodiment, a region of the workflow webpage displays disease awareness information 550 that a physician may select to access updated information relevant to a patient's condition. A workflow webpage may also display informational content areas 570, 580 integrated into the provider-patient appointment calendar 590. Presentation of such information within the workflow reduces the need for external searches and improves clinical efficiency, in one embodiment.

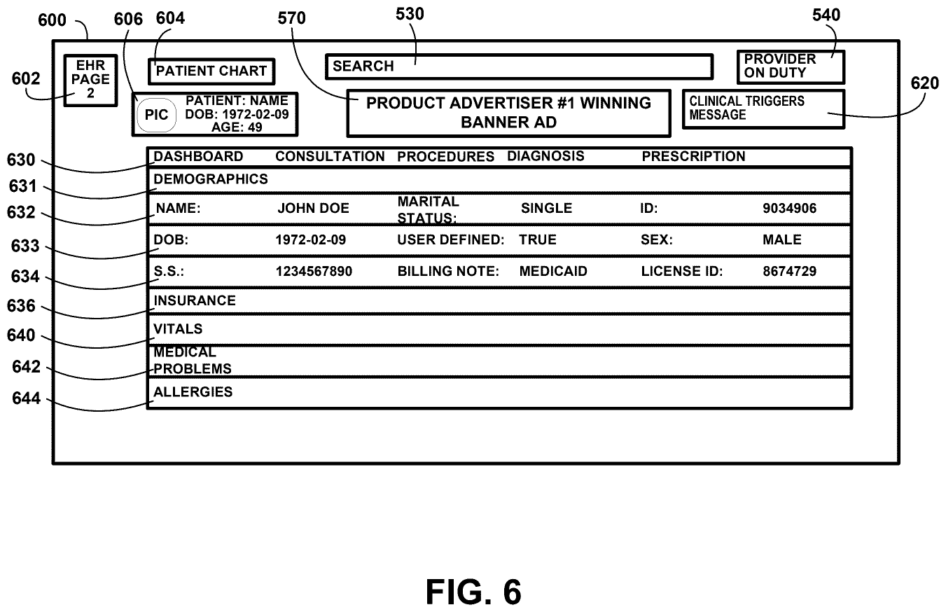

FIG. 6 shows for illustrative purposes only an example of a patient chart of one embodiment. FIG. 6 shows, for example, a physician continuing workflow webpage 600 corresponding to EHR page 2 602. During a patient encounter, a physician may enter and review data on a patient chart 604. Information associated with clinical triggers for a patient is displayed, including patient name, date of birth, age, and patient image 606. The search interface 530 allows a physician on duty 540 to retrieve contextually relevant messages.

In one embodiment, a message interface 620 lists clinical triggers used to select condition-specific information for presentation. The workflow webpage includes selectable modules such as dashboard, consultation, procedures, diagnosis, and prescription functions 630. The patient chart 604 displays patient demographic data 631 including name, marital status, identification number 632, date of birth, and sex 633. Additional demographic data includes billing information and license identification 634. Additional patient data available for clinical review includes insurance information 636, vital signs 640, medical problems 642, and allergy information 644, in one embodiment.

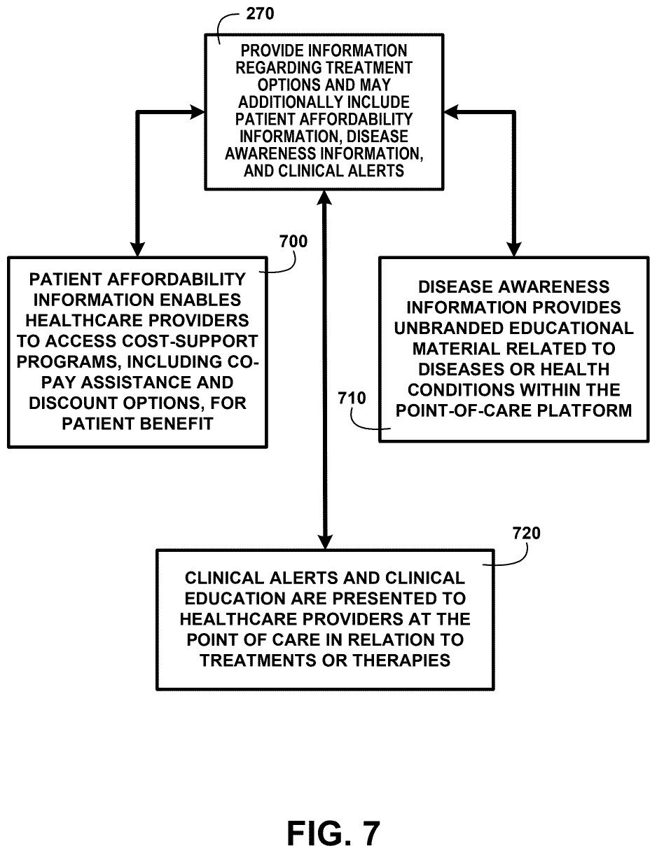

FIG. 7 shows a block diagram of an overview of providing a physician with information of one embodiment. FIG. 7 illustrates the delivery of patient affordability information, disease awareness information, and clinical alerts 270 within an EHR workflow. Patient affordability information enables healthcare providers to access cost-support programs, including co-pay assistance and discount options, for patient benefit 700. Disease awareness information provides unbranded educational material related to diseases or health conditions within the point-of-care platform 710. Clinical alerts and clinical education are presented to healthcare providers at the point of care in relation to treatments or therapies 720, in one embodiment.

In certain embodiments, the system comprises one or more processing subsystems including one or more processors, graphics processing units (GPUs), tensor processing units (TPUs), digital signal processors (DSPs), neural processing units (NPUs), or other specialized processing circuitry configured to execute machine-readable instructions stored in one or more memory devices. The memory devices may include volatile memory, non-volatile memory, flash memory, solid-state storage, or other storage media configured to store executable instructions, intermediate data, feature representations, trained model parameters, and historical datasets. The processing subsystems may be configured to perform operations including data acquisition, signal preprocessing, feature generation, data transformation, model execution, inference, decision-making, and output generation.

In certain embodiments, the system further comprises one or more data storage systems or databases configured to store structured and unstructured data, including raw sensor data, processed data, feature vectors, model outputs, metadata, user-specific data, and system-level data. The databases may be implemented using relational databases, non-relational databases, distributed databases, data lakes, or other storage architectures, and may support indexing, querying, and retrieval operations to facilitate efficient data processing.

In certain embodiments, the system includes one or more artificial intelligence or machine learning components configured to process structured data and generate output data. The machine learning components may include one or more trained models implemented using a variety of model architectures, including but not limited to linear regression models, logistic regression models, decision tree models, random forest models, gradient boosting models (including XGBoost or similar implementations), support vector machines, Bayesian inference models, probabilistic graphical models, hidden Markov models, k-nearest neighbor models, or ensemble models combining multiple model types.

In certain embodiments, the system may utilize neural network-based architectures, including feedforward neural networks, convolutional neural networks (CNNs) for spatial or image-based processing, recurrent neural networks (RNNs) for sequential data processing, long short-term memory (LSTM) networks, gated recurrent unit (GRU) networks, transformer-based architectures utilizing attention mechanisms, attention-based sequence models, graph neural networks (GNNs), autoencoders and variational autoencoders (VAEs) for representation learning, generative adversarial networks (GANs) for data generation, and hybrid or ensemble architectures combining multiple neural network types. In certain embodiments, models may operate on different data modalities or feature subsets and may be arranged in parallel, sequential, hierarchical, or ensemble configurations to generate intermediate and final outputs.

In certain embodiments, the system may include training and updating mechanisms for machine learning models. Training may be performed using labeled datasets in supervised learning frameworks, unlabeled datasets in unsupervised learning frameworks, partially labeled datasets in semi-supervised learning frameworks, or feedback-driven approaches in reinforcement learning frameworks. Training processes may include data preprocessing, feature engineering, model parameter optimization, loss function minimization, regularization, cross-validation, and performance evaluation. In certain embodiments, training may be performed locally on a device, remotely on a server or cloud computing platform, or using distributed computing techniques such as federated learning, in which model updates are generated across multiple devices without centralized aggregation of raw data.

In certain embodiments, inference operations may be performed in real time, near real time, or batch processing modes. The system may dynamically allocate computational tasks between local devices and remote computing systems based on latency requirements, computational resource availability, bandwidth constraints, or application-specific requirements. In certain embodiments, edge computing devices may perform initial data processing and feature extraction, while cloud-based systems perform more computationally intensive model execution and analysis.

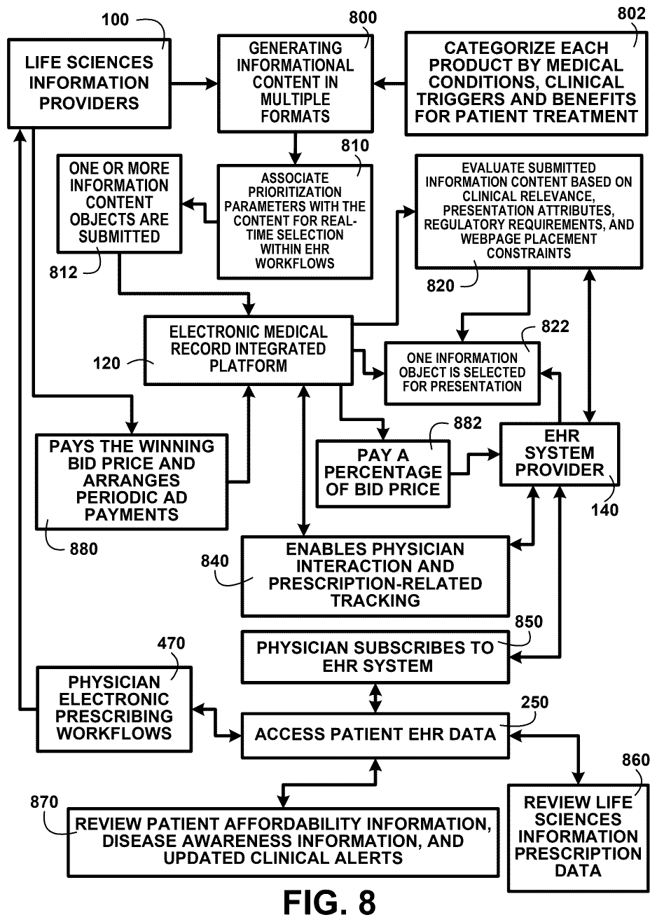

FIG. 8 shows a block diagram of an overview of the creation of information content in various formats of one embodiment. FIG. 8 shows life sciences information providers 100 generating informational content in multiple formats 800. Life sciences information providers 100 categorize information objects by medical conditions, clinical triggers, and therapeutic benefits relevant to patient treatment 802. The information providers further associate prioritization parameters with the content for real-time selection within EHR workflows 810. One or more information content objects 812 are submitted to an electronic medical record integrated platform 120.

The electronic medical record integrated platform 120 and an EHR system provider 140 evaluate submitted information content based on clinical relevance, presentation attributes, regulatory requirements, and webpage placement constraints 820. Following the evaluation, at least one information object is selected for presentation 822. In one embodiment, upon selection of the winning bid, the life sciences information provider pays the winning bid price, arranges periodic advertising payments 880, and pays a percentage of the bid price 882 rated platform 120 or an EHR system provider 140 in accordance with predefined commercial terms.

The EHR system provider 140 processes webpage layout configuration, validates information links, and enables physician interaction and prescription-related tracking 840. A physician subscribes to an EHR system 850 to access patient EHR data 250 and review life sciences information, including prescription-related data 860.

The physician may further review patient affordability information, disease awareness information, and updated clinical alerts 870. The presented information supports physician electronic prescribing workflows 470. In one embodiment, system-level operational and administrative processes associated with information delivery are managed by the electronic medical record integrated platform 120 in coordination with the EHR system provider 140.

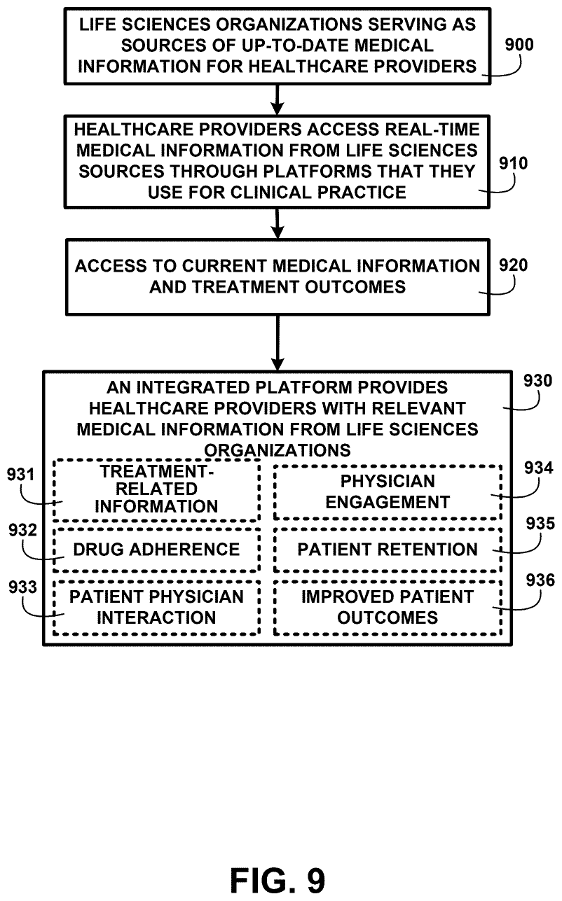

FIG. 9 shows a block diagram of an overview of providing the latest medical information to healthcare providers of one embodiment. FIG. 9 shows life sciences organizations serving as sources of up-to-date medical information for healthcare providers 900. Healthcare providers access real-time medical information from life sciences sources through platforms that they use for clinical practice 910. The physician does not need to conduct independent research, as the EHR system provides access to current medical information and treatment outcomes 920.

An integrated platform provides healthcare providers with relevant medical information from life sciences organizations 930. The delivered information includes treatment-related information 931 and supports improved drug adherence 932, enhances patient-physician interactions 933, improves physician engagement 934, and supports patient retention 935, collectively contributing to improved patient outcomes 936.

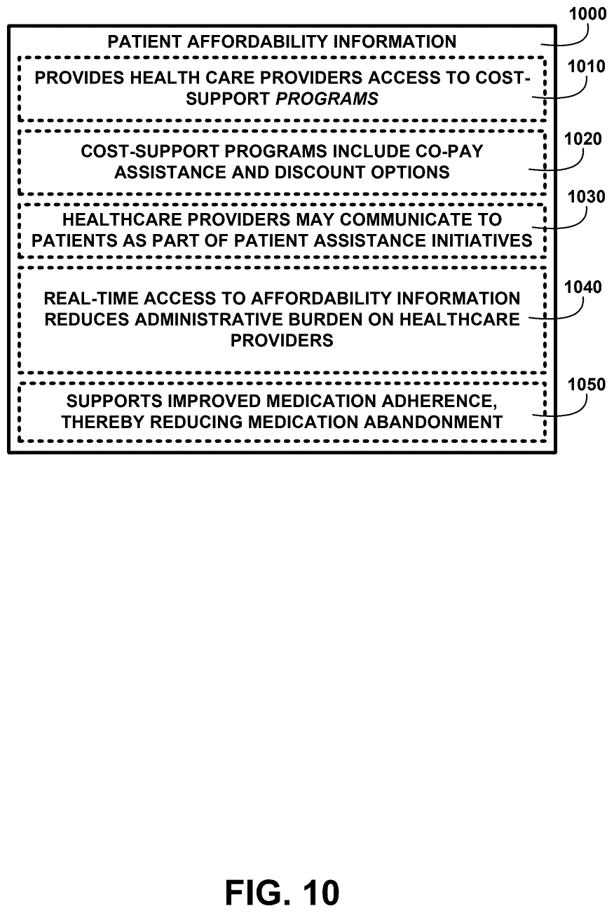

FIG. 10 shows a block diagram of an overview of patient affordability information of one embodiment. FIG. 10 shows patient affordability information 1000 that provides healthcare providers with access to cost-support programs 1010. The cost-support programs include co-pay assistance and discount options 1020 that healthcare providers may communicate to patients as part of patient assistance initiatives 1030. Real-time access to affordability information reduces administrative burden on healthcare providers 1040 and supports improved medication adherence, thereby reducing medication abandonment 1050, in one embodiment.

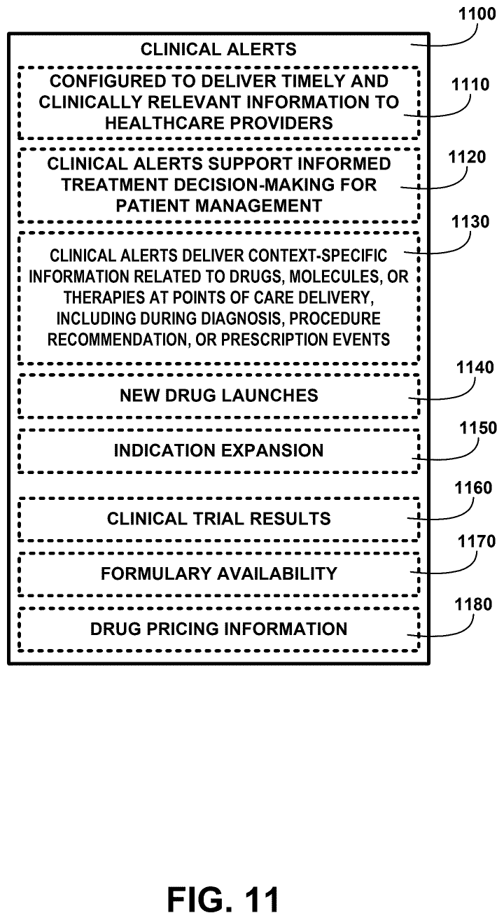

FIG. 11 shows a block diagram of an overview of clinical alerts of one embodiment. FIG. 11 shows clinical alerts 1100 configured to deliver timely and clinically relevant information to healthcare providers 1110. The clinical alerts support informed treatment decision-making for patient management 1120 without requiring independent research. Clinical alerts deliver context-specific information related to drugs, molecules, or therapies at the point of care, including during diagnosis, procedure recommendation, or prescription events 1130. The alerts may include information related to new drug launches 1140, indication expansions 1150, clinical trial results 1160, formulary availability 1170, and drug pricing information 1180, in one embodiment.

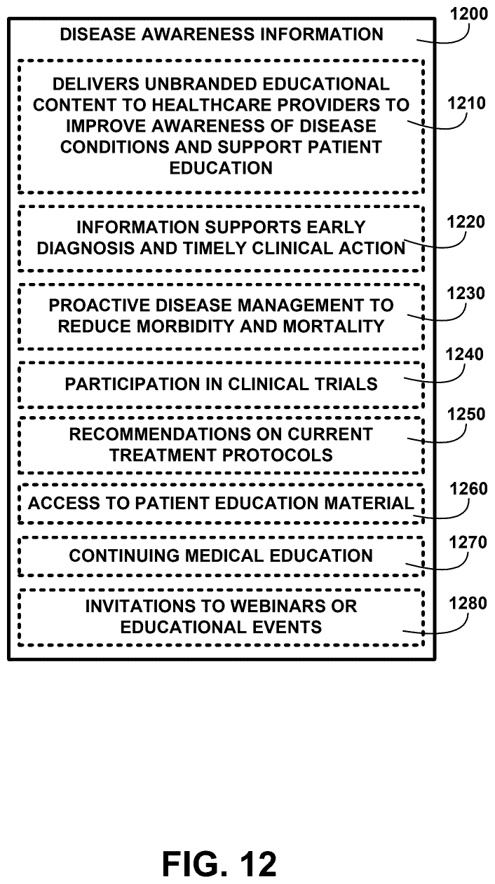

FIG. 12 shows a block diagram of an overview of disease awareness information of one embodiment. FIG. 12 shows disease awareness information 1200 that delivers unbranded educational content to healthcare providers to improve awareness of disease conditions and support patient education 1210. The information supports early diagnosis and timely clinical action 1220 and promotes proactive disease management to reduce morbidity and mortality 1230. In one embodiment, a physician may determine that a patient is a candidate for participation in clinical trials 1240. The information further provides recommendations regarding current treatment protocols 1250. Patients benefit from enhanced engagement through access to educational materials 1260, while physicians are supported through readily available continuing medical education resources 1270 and invitations to webinars or educational events 1280, in one embodiment.

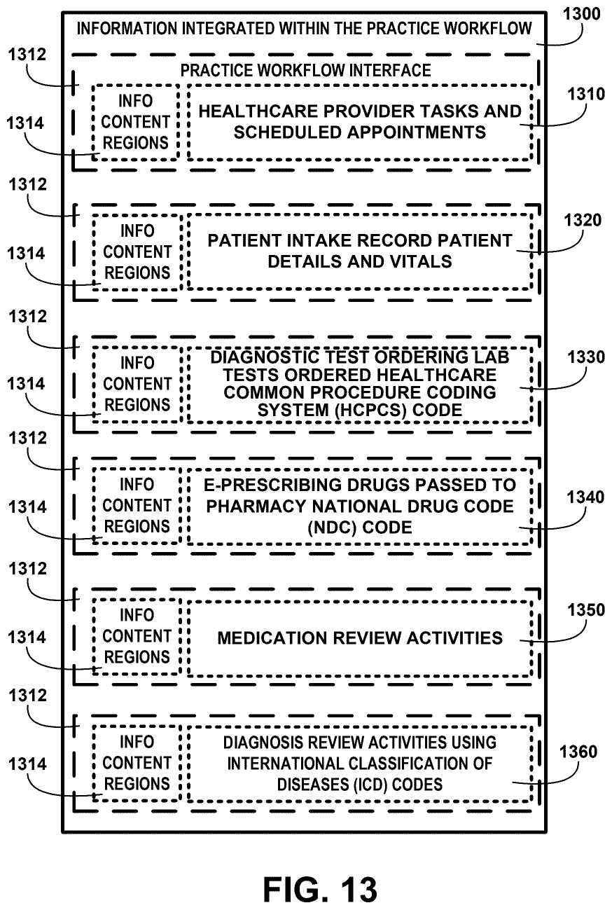

FIG. 13 shows a block diagram of an overview of information integrated within a clinical practice workflow of one embodiment. FIG. 13 illustrates informational content integrated within the practice workflow 1300. An initial workflow webpage may display healthcare provider tasks and scheduled appointments 1310 on a practice workflow interface 1312, with one or more informational content regions 1314 presented therein. Patient intake processes record patient details and vital signs 1320 on the same practice workflow interface 1312. Informational content regions 1314 are maintained within the workflow display.

Diagnostic test ordering, including laboratory test orders associated with Healthcare Common Procedure Coding System (HCPCS) codes 1330, is performed within the practice workflow interface 1312, while informational content regions 1314 remain visible. Electronic prescribing workflows transmit prescription data to a pharmacy using National Drug Code (NDC) identifiers 1340 within the same interface. Medication review activities 1350 and diagnosis review activities using International Classification of Diseases (ICD) codes 1360 are similarly conducted within the practice workflow interface 1312, with informational content integrated throughout the workflow, in one embodiment.

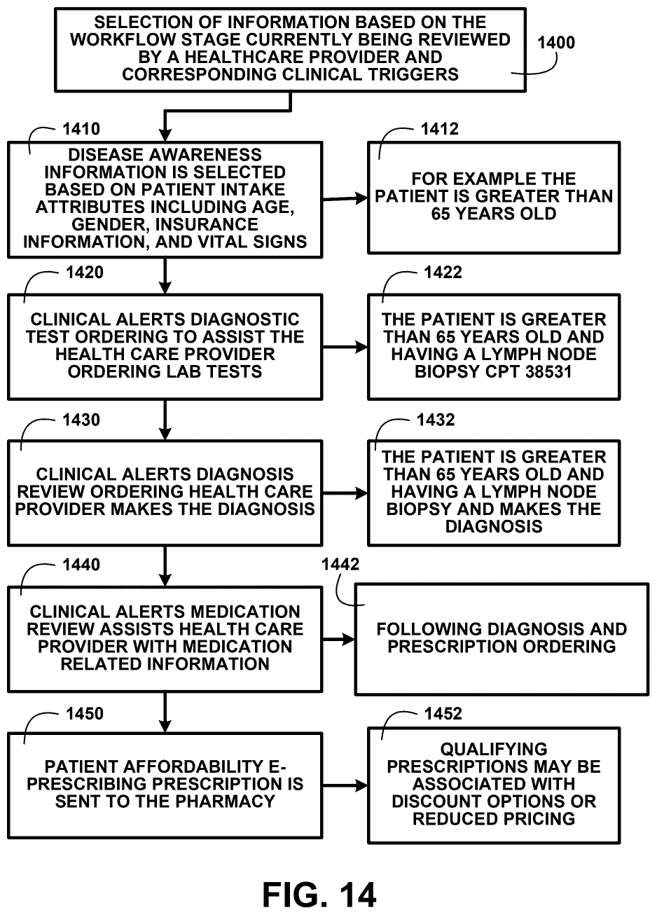

FIG. 14 shows a block diagram of an overview of selecting context-specific information based on a clinical workflow of one embodiment. FIG. 14 illustrates the selection of information based on the workflow stage currently being reviewed by a healthcare provider and corresponding clinical triggers 1400. In one embodiment, disease awareness information is selected based on patient intake attributes, including age, gender, insurance information, and vital signs 1410, for example, where a patient is greater than sixty-five years of age 1412.

One workflow interface includes clinical alerts associated with diagnostic test ordering to assist a healthcare provider when ordering laboratory tests 1420. In one embodiment, the workflow reflects that the patient is greater than sixty-five years of age and undergoing a lymph node biopsy 1422. Clinical alerts associated with diagnosis review assist the healthcare provider during diagnostic determination 1430. In this embodiment, the workflow reflects that the patient is greater than sixty-five years of age, has undergone a lymph node biopsy, and a diagnosis has been made 1432. Clinical alerts associated with medication review assist the healthcare provider with medication-related information 1440. In one embodiment, following diagnosis and prescription ordering 1442, patient affordability information is presented during electronic prescribing, and prescription data is transmitted to a pharmacy 1450, and qualifying prescriptions may be associated with discount options or reduced pricing 1452 of one embodiment.

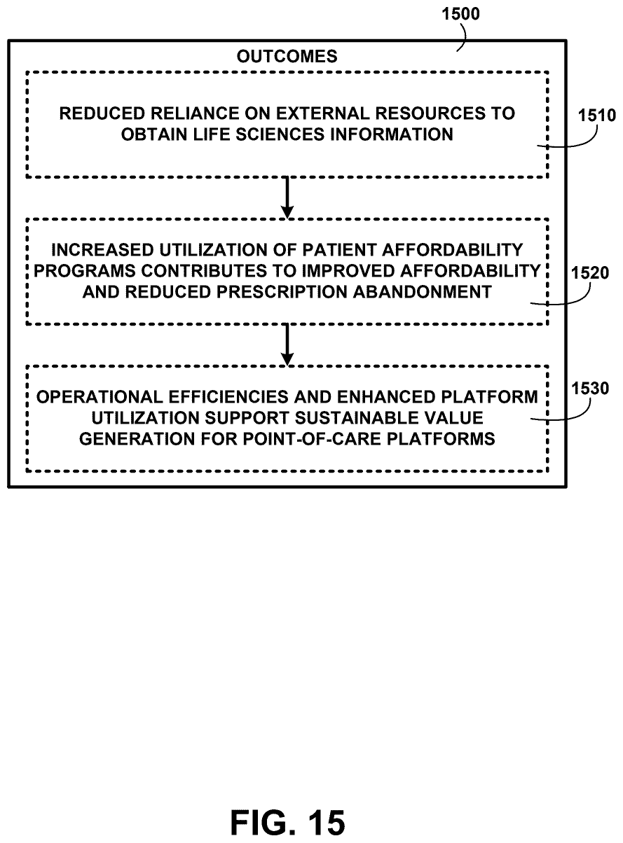

FIG. 15 shows a block diagram of an overview of outcomes of one embodiment. FIG. 15 illustrates outcomes 1500 that benefit healthcare providers. In one embodiment, an outcome includes reduced reliance on external resources to obtain life sciences information 1510. In another embodiment, increased utilization of patient affordability programs contributes to improved affordability and reduced prescription abandonment 1520. In further embodiments, operational efficiencies and enhanced platform utilization support sustainable value generation for point-of-care platforms 1530, in one embodiment.

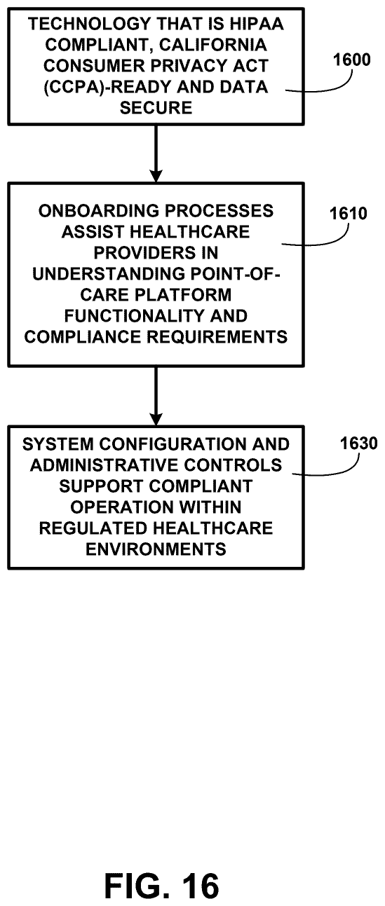

FIG. 16 shows a block diagram of an overview of regulatory compliance of one embodiment. FIG. 16 illustrates technology configured to operate in compliance with the Health Insurance Portability and Accountability Act (HIPAA), the California Consumer Privacy Act (CCPA), and applicable data security standards 1600. Onboarding processes assist healthcare providers in understanding point-of-care platform functionality and compliance requirements 1610. System configuration and administrative controls support compliant operation within regulated healthcare environments 1630, in one embodiment.

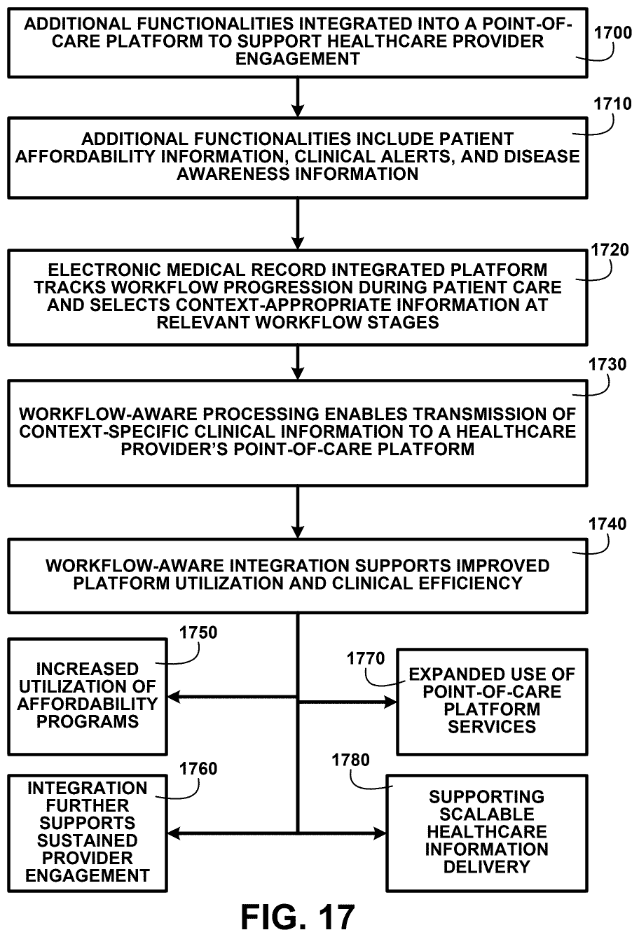

FIG. 17 shows a block diagram of an overview of engaging with healthcare providers of one embodiment. FIG. 17 illustrates additional functionalities integrated into a point-of-care platform to support healthcare provider engagement 1700.

The additional functionalities include patient affordability information, clinical alerts, and disease awareness information 1710. An electronic medical record integrated platform tracks workflow progression during patient care and selects context-appropriate information at relevant workflow stages 1720. Workflow-aware processing enables transmission of context-specific clinical information to a healthcare provider's point-of-care platform 1730. The workflow-aware integration supports improved platform utilization and clinical efficiency 1740 and increased utilization of affordability programs 1750. The integration further supports sustained provider engagement 1760 and expanded use of point-of-care platform services 1770, thereby supporting scalable healthcare information delivery 1780, in one embodiment.

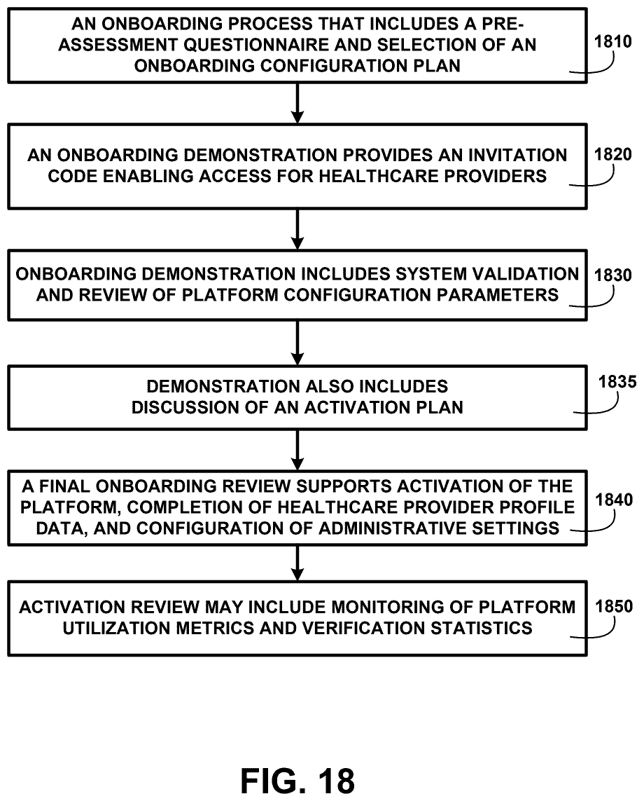

FIG. 18 shows a block diagram of an overview of an onboarding process of one embodiment. FIG. 18 illustrates an onboarding process that includes a pre-assessment questionnaire and selection of an onboarding configuration plan 1810. An onboarding demonstration provides an invitation code enabling access for healthcare providers 1820. The onboarding demonstration includes system validation and review of platform configuration parameters 1830. The onboarding demonstration further provides guidance regarding information placement regions, display formats, and workflow integration considerations. The demonstration also includes a discussion of an activation plan 1835. A final onboarding review supports activation of the platform, completion of healthcare provider profile data, and configuration of administrative settings 1840. The activation review may include monitoring of platform utilization metrics and verification statistics 1850, in one embodiment.

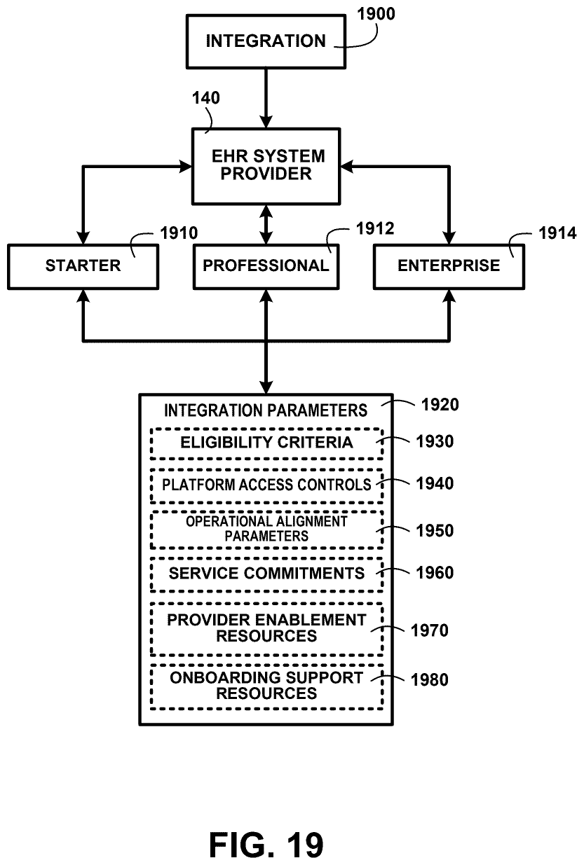

FIG. 19 shows a block diagram of an overview of platform integration and partner configuration of one embodiment. FIG. 19 illustrates integration 1900 between the electronic medical record integrated platform 120 of FIG. 1 and an EHR system provider 140. Integration levels may be categorized based on subscriber scale as starter 1910, professional 1912, or enterprise 1914 tiers. Integration parameters 1920 vary by configuration level and may include eligibility criteria 1930, platform access controls 1940, operational alignment parameters 1950, service commitments 1960, provider enablement resources 1970, and onboarding support resources 1980, in one embodiment.

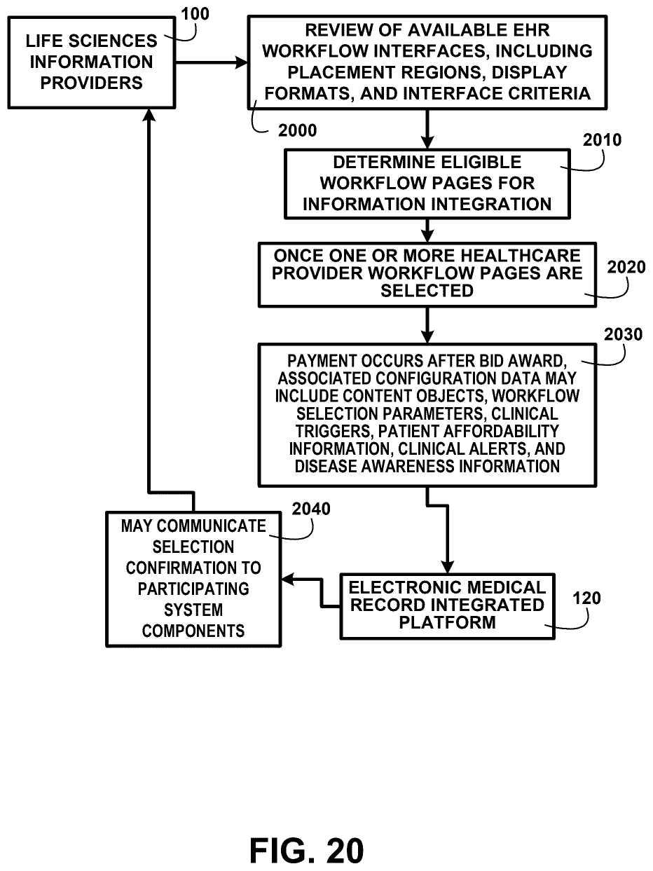

FIG. 20 shows a block diagram of an overview of the workflow page configuration of one embodiment. FIG. 20 illustrates a review of available life sciences information providers 100, EHR workflow interfaces, including placement regions, display formats, and interface criteria 2000, to determine eligible workflow pages for information integration 2010. Once one or more healthcare provider workflow pages 2020 are selected, payment occurs after bid award, and associated configuration data may include content objects, workflow selection parameters, clinical triggers, patient affordability information, clinical alerts, and disease awareness information 2030. An electronic medical record integrated platform 120 may communicate selection confirmation 2040 to participating system components of one embodiment.

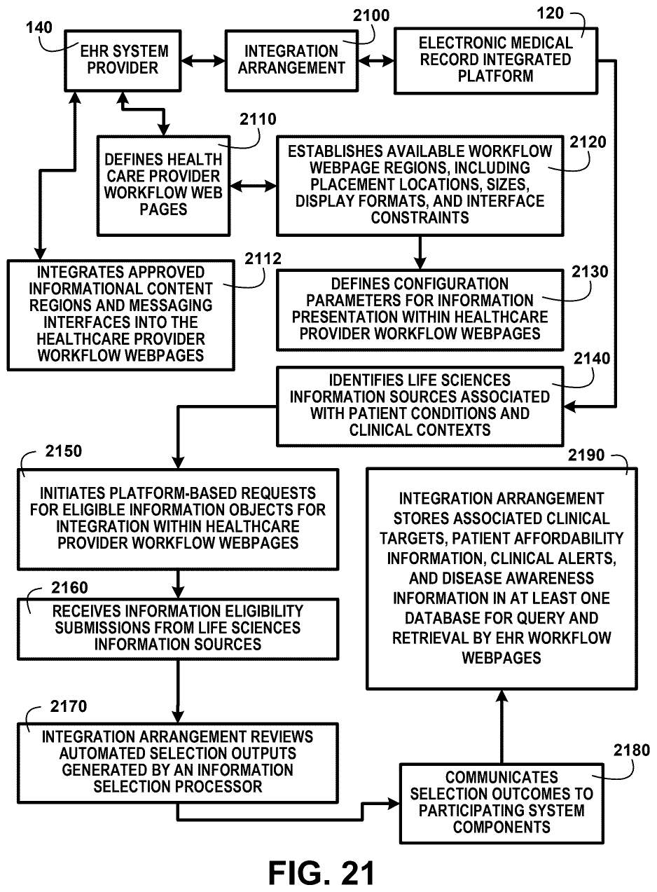

FIG. 21 shows a block diagram of an overview of establishing healthcare provider workflow webpages of one embodiment. FIG. 21 illustrates an integration arrangement 2100 between an EHR system provider 140 and the electronic medical record integrated platform 120. An EHR system provider 140 defines healthcare provider workflow webpages 2110 and integrates approved informational content regions and messaging interfaces into the healthcare provider workflow webpages 2112.

The EHR system provider 140 establishes available workflow webpage regions, including placement locations, sizes, display formats, and interface constraints 2120. The integration arrangement defines configuration parameters for information presentation within healthcare provider workflow webpages 2130. The electronic medical record integrated platform 120 identifies life sciences information sources associated with patient conditions and clinical contexts 2140. An electronic medical record integrated platform 120 initiates platform-based requests for eligible information objects for integration within healthcare provider workflow webpages 2150.

The electronic medical record integrated platform 120 receives information eligibility submissions from life sciences information sources 2160. The integration arrangement reviews automated selection outputs generated by an information selection processor 2170. The electronic medical record integrated platform 120 communicates selection outcomes to participating system components 2180. The integration arrangement stores associated clinical targets, patient affordability information, clinical alerts, and disease awareness information in at least one database for query and retrieval by EHR workflow webpages 2190, in one embodiment.

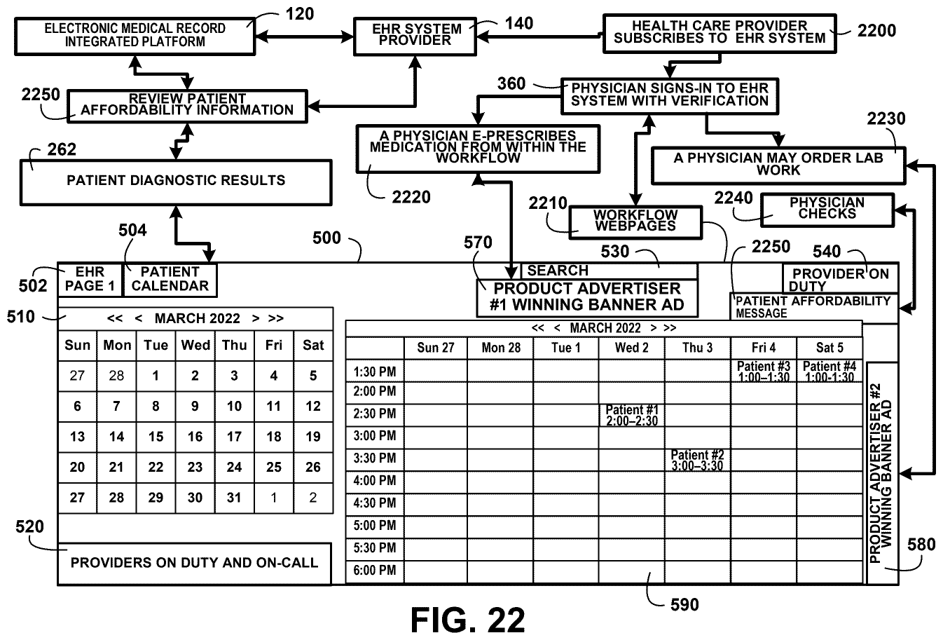

FIG. 22 shows a block diagram of an overview of physician electronic prescribing within an integrated EHR workflow of one embodiment. FIG. 22 illustrates the electronic medical record integrated platform 120 and the EHR system provider 140 operating as integrated systems to support point-of-care workflows. A healthcare provider subscribes to an EHR system 2200 and signs in using verified credentials 360 to access workflow webpages 2210. In one embodiment, a physician electronically prescribes medication based on information presented within the workflow 2220.

A physician checks 2240 and may order laboratory work 2230 and review patient affordability information 2250 to obtain updated cost-support information. In one embodiment, a physician workflow webpage 500 corresponding to EHR page 1 502 displays a patient calendar 504, a current month calendar 510, provider listings 520, and a search interface 530. A physician on duty 540 may interact with informational content regions 570, 580 to obtain additional clinical or product-related information. The provider-patient appointment calendar 590 transmits clinical trigger data, including patient diagnostic results 262, as part of coordinated EHR system processing 2250, in one embodiment.

The embodiments described herein may be implemented using combinations of hardware, software, firmware, or microcode, and may be embodied in one or more non-transitory computer-readable media storing instructions that, when executed by one or more processors, processing circuits, or computing systems, cause the system to perform the operations described herein. The hardware components described herein may include discrete components, integrated circuits, application-specific integrated circuits (ASICs), field-programmable gate arrays (FPGAs), system-on-chip (SoC) architectures, or distributed computing components arranged across multiple devices. The processing operations described herein may be implemented using dedicated circuitry, programmable logic, or combinations of hardware and software executed by general-purpose or specialized processors.

It will be understood that the described systems may operate across heterogeneous computing environments including combinations of wearable devices, mobile devices, smartphones, augmented reality (AR) devices, virtual reality (VR) devices, mixed reality (MR) devices, IoT devices, edge computing devices, servers, and cloud computing platforms. Communication between such devices may occur using a variety of communication protocols, network topologies, and addressing schemes, including packet-based communication, streaming protocols, publish-subscribe architectures, or peer-to-peer communication frameworks. In certain embodiments, devices may be uniquely identified and addressed using identifiers such as IP addresses, MAC addresses, device identifiers, or cryptographic identifiers.

Input devices and sensors may include any devices capable of generating signals representative of physical, environmental, biological, user, or object states, and such signals may include electrical signals, optical signals, acoustic signals, electromagnetic signals, or digital representations thereof. The system may further include computer vision and object recognition components configured to process image, video, or sensor data to detect, classify, track, or identify objects, persons, gestures, or environmental features using feature extraction techniques, pattern recognition algorithms, or machine learning models.

The arrangement of components, data structures, processing steps, models, and system architectures described herein is provided for purposes of illustration and enabling disclosure, and alternative configurations, combinations, substitutions, and modifications may be implemented without departing from the scope of the invention. The described embodiments are intended to encompass implementations in which input data is acquired from one or more physical sources, transformed into structured data representations, processed using computational models, and used to generate outputs that correspond to a practical application, technical improvement, or control of a system or device. Accordingly, the scope of the invention is defined by the claims and is not limited to the specific embodiments described herein.

The foregoing has described the principles, embodiments, and modes of operation of the present invention. The invention should not be construed as being limited to the particular embodiments discussed. The above-described embodiments are illustrative rather than restrictive, and variations may be made by those skilled in the art without departing from the scope of the present invention as defined by the following claims.

What is claimed is:

1. A system for integrating regulated life sciences information within an electronic health record (EHR) workflow, comprising:
one or more processors;
one or more non-transitory memory devices storing executable instructions;
an interface configured to communicate with at least one EHR system;
wherein execution of the instructions by the one or more processors causes the system to:
authenticate a healthcare provider accessing a patient record within the EHR system;
extract one or more clinical triggers from the patient record during an active clinical workflow;
select, based on the extracted clinical triggers, at least one information object from a plurality of regulated life sciences information objects stored in a database; and
present the selected information object within a predefined region of an EHR workflow interface without interrupting the clinical workflow.

2. The system of claim 1, wherein the clinical triggers comprise at least one of patient age, diagnosis code, laboratory result, medication order, vital sign, insurance attribute, or procedure code.

3. The system of claim 1, wherein the information object comprises at least one of disease awareness information, patient affordability information, clinical alerts, or prescription-related information.

4. The system of claim 1, wherein the information object is selected using rule-based processing executed by the one or more processors.

5. The system of claim 1, wherein the system ranks information objects using a prioritization algorithm information objects based on correlations between extracted clinical triggers and predefined clinical relevance rules, including associations with diagnosis codes, laboratory result ranges, medication orders, or workflow stage indicators.

6. The system of claim 1, further comprising enforcing access controls to ensure the information object complies with applicable healthcare privacy regulations.

7. The system of claim 1, wherein presentation of the information object occurs concurrently with display of patient data within the EHR interface.

8. A distributed electronic health record integration system, comprising:
a platform server comprising one or more processors and memory;
an information repository storing regulated life sciences information objects associated with clinical triggers;
a workflow monitoring module configured to identify a current stage of a healthcare provider's clinical workflow within an EHR system;
a presentation engine configured to render selected information within the EHR interface;
wherein the workflow monitoring module determines the current workflow stage by analyzing EHR navigation context, page identifiers, or transactional events generated during provider interaction, and wherein the platform server synchronizes content selection across multiple EHR sessions or devices associated with the healthcare provider;
wherein the platform server is configured to;
receive clinical trigger data from the EHR system;
determine the current workflow stage of the healthcare provider;
select at least one information object based on both the clinical trigger data and the workflow stage; and
deliver the selected information object for display within the EHR interface at a context-appropriate moment during the workflow.

9. The system of claim 8, wherein the workflow stage corresponds to at least one of patient intake, diagnosis review, test ordering, medication review, or electronic prescribing.

10. The system of claim 8, wherein the presentation engine formats the selected information object by programmatically adapting content attributes to comply with predefined EHR interface schemas, rendering constraints, or application programming interfaces (APIs) enforced by the EHR system.

11. The system of claim 8, wherein the system maintains an audit log of information presentation events.

12. The system of claim 8, wherein the system prevents presentation of information objects by executing compliance rules that disable rendering or transmission of restricted content based on region-specific regulatory flags or jurisdictional compliance parameters stored in the platform server.

13. The system of claim 8, wherein the workflow monitoring module operates during active user interaction with the EHR system by detecting workflow state transitions based on EHR interface events, user input actions, or EHR system callbacks generated while the healthcare provider navigates the patient record.

14. The system of claim 8, wherein multiple information objects are ranked using rule-based clinical relevance scoring, and one or more information objects are selected for presentation based on matching the workflow stage and clinical trigger thresholds.

15. A healthcare information delivery platform, comprising:
one or more servers configured to interface with multiple EHR systems;
a compliance engine configured to enforce healthcare data protection regulations;
a content selection engine configured to select regulated information based on clinical triggers; and
an analytics module configured to record interaction events;
wherein the platform is configured to:
integrate regulated life sciences information into EHR workflows;
ensure patient data is not exposed outside the EHR system;
log presentation and interaction events for compliance verification; and
support delivery of context-specific information without disrupting clinical care activities.

16. The platform of claim 15, wherein the compliance engine enforces requirements of HIPAA, GDPR, or CCPA.

17. The platform of claim 15, wherein the analytics module generates aggregated metrics without storing personally identifiable patient information.

18. The platform of claim 15, wherein the platform supports integration across multiple healthcare provider devices.

19. The platform of claim 15, wherein the regulated information includes unbranded educational content.

20. The platform of claim 15, wherein the platform supports updating information objects without modification to the EHR system.
